(19) United States
(12) Patent Application Publication
(10) Pub. No.: US 2011/0313192 A1
(43) Pub. Date: Dec. 22, 2011

(54) HETEROGENEOUSLY CATALYZED CARBAMATE DISSOCIATION FOR SYNTHESIS OF ISOCYANATES OVER SOLID LEWIS ACIDS

(75) Inventors: Tobias ROSENDAHL, Mannheim (DE); Torsten Mäurer, Lambsheim (DE); Eckhard Stroefer, Mannheim (DE); Axel Franzke, Mannheim (DE)

(73) Assignee: BASF SE, Ludwigshafen (DE)

(21) Appl. No.: 13/163,928

(22) Filed: Jun. 20, 2011

Related U.S. Application Data
(60) Provisional application No. 61/357,107, filed on Jun. 22, 2010.

(30) Foreign Application Priority Data
Jun. 22, 2010 (EP) 10166781.4

(51) Int. Cl. C07C 263/04

(52) U.S. Cl. 560/345

(57) ABSTRACT

Proposed is a process for preparing isocyanates by dissociating the corresponding carbamates, which comprises dissociating the carbamates in the presence of a heterogeneous Lewis-acidic catalyst.

This patent application claims the benefit of pending U.S. provisional patent application Ser. No. 61/357,107 filed Jun. 22, 2010 incorporated in its entirety herein by reference

The invention relates to a process for preparing isocyanates by dissociation of the corresponding carbamates.

Carbamate dissociation is increasingly gaining in importance as a phosgene-free process for preparing isocyanates. Various apparatuses have been proposed for the technical implementation of carbamate dissociation, more particularly columns (in EP 0 795 543), fluidized-bed reactors (in EP 555 628 and in DE 199 07 648), falling-film or thin-film evaporators (in EP 0 092 738). The carbamate dissociation can be operated in the liquid phase or in the gas phase.

A problem in the thermal dissociation of carbamates is the formation of high molecular weight secondary components which are formed by onward reaction of the dissociation products with themselves or with starting materials. These secondary components may form deposits in the apparatuses, thereby restricting continuous operation and leading to losses of yield. The residues contain, in particular, allophanates and isocyanurates. The byproducts also come about through the reaction of monourethanes (semicarbamates, i.e., a difunctional compound containing one urethane function and one isocyanate function; intermediates in the dissociation of bisurethanes) with themselves. They additionally come about through the reaction of the desired end product with the reactant material.

It was an object of the invention, accordingly, to provide an improved process for preparing isocyanates by dissociating carbamates, with improvements more particularly in the space-time yield and in the selectivity of the carbamate dissociation.

The object is achieved by means of a process for preparing isocyanates by dissociating the corresponding carbamates, which comprises dissociating the carbamates in the presence of a heterogeneous Lewis-acidic catalyst.

It has been found that Lewis-acidic heterogeneous catalysts increase the reaction rate of the carbamate dissociation by a factor of up to 80 as compared with the purely thermal dissociation, with a high selectivity, of more than 90%, of the dissociation to form the corresponding isocyanates.

It has surprisingly been found that these high rate increases and selectivities are achieved only with catalysts which are Lewis-acidic, but not with Brönsted acids, which lead to unwanted secondary reactions in the course of dissociation.

In the process of the invention it is possible to use customary carbamates (also referred to as carbamic esters or urethanes), preferably biscarbamates and polycarbamates, for the dissociation. These carbamates are typically based on the familiar reaction of amines, preferably of diamines or polyamines, more preferably of diamines, with urea and at least one alcohol.

Suitable alcohols for preparing the carbamates include in principle all aliphatic alcohols. It is preferred to select those whose boiling points are sufficiently different from the boiling point of the isocyanates, in order to ensure optimum separation. For preparing the carbamate it is particularly preferred to use aliphatic monohydroxy alcohols having 1 to 4 C atoms per molecule, i.e., methanol, ethanol, propanol, isopropanol, n-butanol and/or isobutanol. Preference extends to alcohols having at least one oxygen heteroatom, more particularly 2-methoxyethanol, 2-ethoxyethanol, 2-propoxyethanol, 2-butoxyethanol, 2-methoxy-1-propanol and/or 1-methoxy-2-propanol.

Amines used are preferably 2,4- and/or 2,6-tolylenediamine (TDA), 2,2′-, 2,4′- and/or 4,4′-diaminodiphenylmethane (MDA) and/or higher homologs (polyphenylenepolymethylenepolyamines, pMDA), 1,6-hexamethylenediamine (HDA), 1-amino-3,3,5-trimethyl-5-aminomethylcyclohexane (also referred to below as isophoronediamine, IPDA), 1,5- and/or 1,8-diaminonaphthalene, 4,4′-diaminobiphenyl, 1,3- and/or 1,4-diaminobenzene, 2,4- and/or 2,6-hexahydrotoluylenediamine and/or 4,4′-, 2,4′- and/or 2,2′-dicyclohexylmethanediamine. The structures of the amines used determine the structures of the isocyanates obtainable in accordance with the thermal dissociation. With particular preference the urethanes used are based on 2,4- and/or 2,6-tolylenediamine (TDA), 2,2′-, 2,4′- and/or 4,4′-diaminodiphenylmethane (MDA) and/or higher homologs (polyphenylenepolymethylenepolyamines, pMDA), 1,6-hexamethylenediamine (HDA), isophoronediamine (IPDA) and/or 1,5-diaminonaphthalene as amine component, and on methanol, n-propanol, isopropanol, n-butanol or in particular, isobutanol or 2-methoxyethanoi as alcohol.

In particular, accordingly, the following diurethanes or polyurethanes are used for the dissociation: 2,4- and/or 2,6-tolylene diisobutylurethane, 2,4- and/or 2,6-tolylenedimethoxyethylurethane, 2,4- and/or 2,6-tolylenedipropylurethane, 2,4- and/or 2,6-tolylenedimethylurethane, 1,5-naphthylenediisobutylurethane, 1,5-naphthylenedimethoxyethylurethane, 1,5-naphthylenedipropylurethane, 1,5-naphthylenedimethylurethane, 4,4′-, 2,4′- and/or 2,2′-diphenylmethane-diisobutylurethane, 4,4′-, 2,4′- and/or 2,2′-diphenylmethanedimethoxyethylurethane, 4,4′-, 2,4′- and/or 2,2′-diphenylmethanedipropylurethane, 4,4′-, 2,4′- and/or 2,2′-diphenylmethanedimethylurethane, polyphenylenepolymethylenepolymethoxyethylurethane, polyphenylenepolymethylenepolymethylurethane, polyphenylenepolymethylenepolypropylurethane, polyphenylenepolymethylenepolyisobutylurethane, 1,6-hexamethylenediisobutylurethane, 1,6-hexamethylenedimethoxyethylurethane, 1,6-hexamethylenedipropylurethane, 1,6-hexamethylenedimethylurethane, isophoronediisobutylurethane, isophoronedimethoxyethyiurethane, isophoronedipropylurethane and/or isophoronedimethyfurethane, and mixtures of the stated urethanes as well may be used for the dissociation.

With particular preference, the following isocyanates are prepared by thermal dissociation of the corresponding diurethanes: 2,4- and/or 2,6-tolylene diisocyanate (TDI), 2,2′-, 2,4′- and/or 4,4′-diphenylmethane diisocyanate (MDI, polyphenylenepolymethylene polyisocyanates, p-MDI), 1,6-hexamethylene diisocyanate (HDI), 1-amino-3,3,5-trimethyl-5-aminomethylcyclohexane diisocyanate (isophorone diisocyanate, PDI) and/or 1,5-diisocyanatonaphthalene (NDI).

The heterogeneous Lewis-acidic catalyst used in accordance with the invention is preferably a supported catalyst which comprises a ceramic support to which a metal has been applied.

Ceramic supports found suitable for use in accordance with the invention include more particularly zeolites, spinels and perovskites. Particularly suitable are zeolites, especially Na—Y-zeolites.

Suitable metals applied to the support are particularly zinc, aluminum, and iron. Zinc is applied preferably as a metal to the ceramic support.

The heterogeneous Lewis-acidic catalyst may be prepared preferably by solid-state reaction of ZnCl₂ and Na—Y-zeolite by heat treatment or with microwave irradiation. Processes of this kind are described in, for example, Journal of Molecular Catalysis A: Chemical 209 (2004) pages 171 to 177.

In one preferred embodiment, the Lewis-acidic heterogeneous catalyst may be used in the form of a suspended catalyst.

In another preferred embodiment, the Lewis-acidic heterogeneous catalyst is used as a coating on the inside wall of a reactor in which the carbamate dissociation is carried out.

The reactor in which the carbamate dissociation is carried out and whose inside wall is coated with the Lewis-acidic catalyst is preferably a falling-film evaporator. The growth of zeolite-based catalysts onto different materials, especially glass or metal, is known from the technical literature.

The conduct of the carbamate dissociation in a falling-film evaporator has the further advantage over carbamate dissociation in the presence of a suspended catalyst that the reaction products of the dissociation, in other words alcohol and isocyanate, can be removed at different points in the reactor, thereby making it possible to prevent the formation of unwanted byproducts by further reaction of said products.

The invention is illustrated below with reference to working examples.

The dissociation investigated was that of 2,4-di-n-propyl-tolylenedimethylurethane (TDU) to the corresponding toluylene diisocyanate (TDI). The catalyst used was an Na—Y-zeolite catalyst treated with zinc chloride. The catalyst was prepared in accordance with the instructions given in Journal of Molecular Catalysis A: Chemical 209 (2004), pages 171 to 177. For this purpose, 10 g of Na—Y-zeolite, having a modulus of 2.8, were ground in an agate mortar with 3.75 g, corresponding to 27.5 mmol, of zinc chloride, and the ground material was then heat-treated at 200° C. for two hours, causing the metal to distribute itself evenly over the surface of the zeolite and to develop the Lewis-acidic surface properties.

Catalyst testing took place in a unit designed for the thermal dissociation of carbamates, comprising a 250 ml four-neck flask, a 600 mm glass column packed with 5×5 mm glass rings, and with heating and stirrer. Serving as a reference were the results of the thermal dissociation. The analysis of the test results took place by means of liquid chromatography (HPLC).

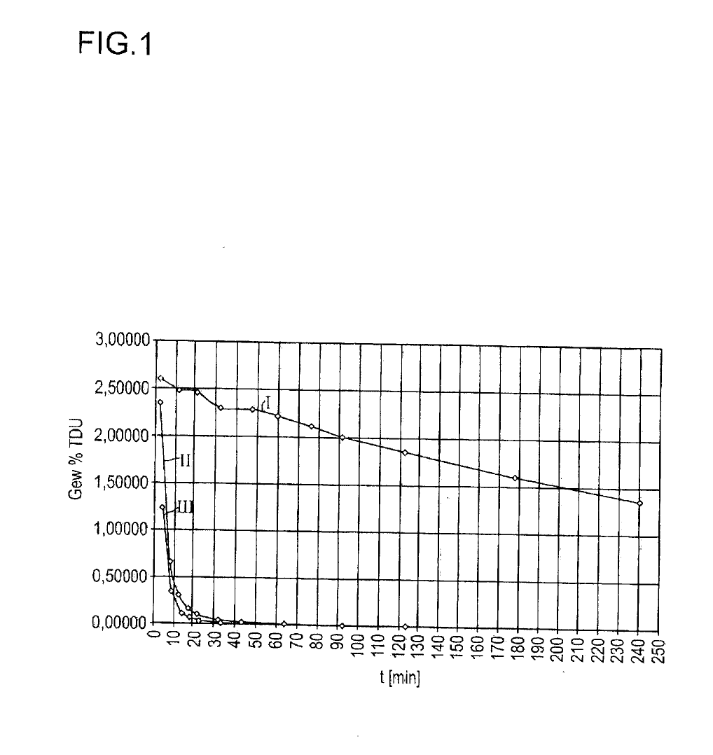

In FIG. 1 below, the reaction profiles have been plotted, in other words the weight decrease, in percent, in the carbamate used over the time, in minutes, with curve I representing the reaction profile for the thermal dissociation, curve II the reaction profile for the dissociation in the presence of 1 mol % of the heterogeneous Lewis-acidic catalyst, and curve III the reaction profile in the presence of 10 mol % of the heterogeneous Lewis-acidic catalyst. The figures show that the reaction rate increases markedly in the presence of the catalyst.

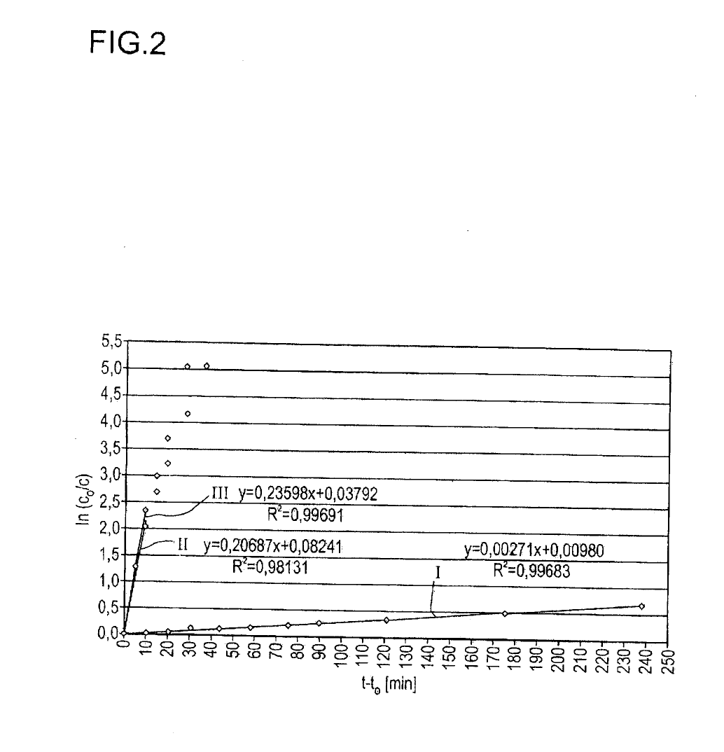

In FIG. 2 below, the rate constants have been shown for the carbamate dissociation from the working examples. The rate constants show an increase by a factor of up to 80 for the catalytic dissociation as compared with the thermal dissociation.

In detail, in the drawing,

FIG. 1 shows in graph form the reaction profiles of the thermal dissociation in comparison to the inventive catalytic dissociation in the presence of a heterogeneous Lewis-acidic zeolite, and

FIG. 2 shows in graph form the reaction constants for the same dissociation reactions as in FIG. 1.

The graph in FIG. 1 shows the decrease in concentration of the TDU reactant, in percent by weight, on the ordinate, over the time t [min], on the abscissa. The top curve I shows the reaction profile for the thermal dissociation, for comparison; curve II shows the reaction profile for the catalytic dissociation in the presence of 1 mol % of zinc-treated Na—Y-zeolite; and the bottom curve, curve III, shows the reaction profile for the catalytic dissociation in the presence of 10 mol % of Na—Y-zeolite. Curves II and III, which illustrate the examples according to the invention, show a marked increase in the reaction rate as compared with the comparative example, curve I, for the purely thermal dissociation.

The graph in FIG. 2 shows the rate constants In (c₀/c), on the ordinate, against the time, t-t₀ [min], on the abscissa, for the thermal dissociation, for comparison (curve I), for the inventive catalytic dissociation in the presence of 1 mol % of zinc-treated Na—Y-zeolite (curve II), and for the inventive catalytic dissociation in the presence of 10 mol % of zinc-treated Na—Y-zeolite (curve III).

1. A process for preparing isocyanates by dissociating the corresponding carbamates, which comprises dissociating the carbamates in the presence of a heterogeneous Lewis-acidic catalyst.

2. The process according to claim 1, wherein the Lewis-acidic catalyst comprises a ceramic support to which a metal has been applied.

3. The process according to claim 2, wherein the ceramic support is a zeolite, a spinel or a perovskite.

4. The process according to claim 3, wherein the ceramic support is a zeolite, preferably a Na—Y-zeolite.

5. The process according to claim 2, wherein the metal applied to the ceramic support is zinc, aluminum or iron.

6. The process according to claim 5, wherein the metal applied to the ceramic support is zinc.

7. The process according to claim 6, wherein the heterogeneous Lewis-acidic catalyst has been prepared by solid-state reaction of ZnCl₂ and Na—Y-zeolite with heat treatment or microwave irradiation.

8. The process according to claim 1, wherein the heterogeneous Lewis-acidic catalyst is used in the form of a suspended catalyst.

9. The process according to claim 1, wherein the heterogeneous Lewis-acidic catalyst is used as a coating on the inside wall of a reactor in which the carbamate dissociation is carried out.

10. The process according to claim 9, wherein the reactor in which the carbamate dissociation is carried out is a falling-film evaporator.
